(19) United States
(12) Plant Patent Application Publication
(10) Pub. No.: US 2002/0188981 P1
(43) Pub. Date: Dec. 12, 2002

(54) Chrysanthemum plant named 'Gedi One Sav'

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,956

(22) Filed: Jun. 4, 2001

(51) Int. Cl. A01H 5/00

(52) U.S. Cl. PLT/292

(57) ABSTRACT

A new and distinct Chrysanthemum plant variety characterized by a large, decorative type inflorescence, consistent flowering response to short days, blooming consistently after 49 days of short day length, free branching habit, and highly floriferous plants.

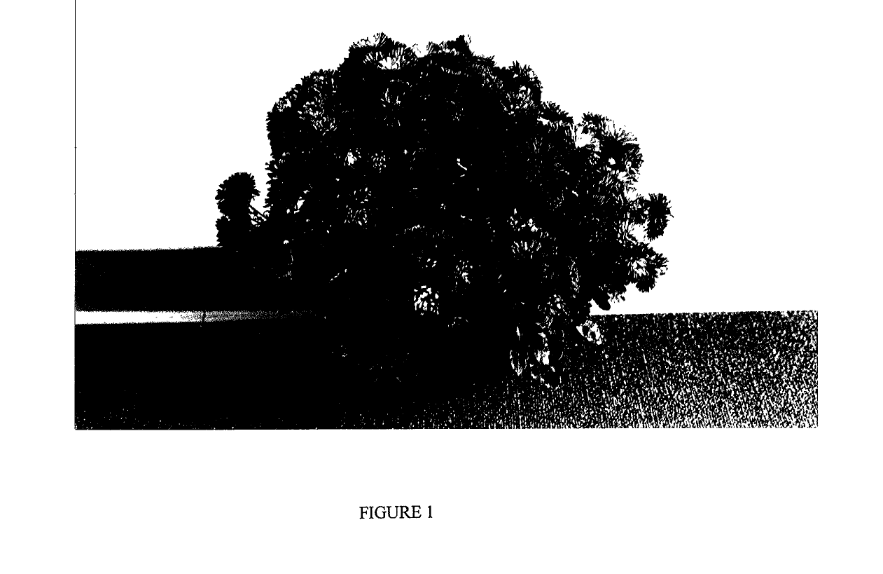

### BACKGROUND OF THE INVENTION

The present invention relates to a new and distinct cultivar of Chrysanthemum plant, botanically known as Dendranthema morifolium and herein after referred to by the cultivar name ‘GEDI ONE SAV.’

This new cultivar originated from hybridization of a female parent, cultivar ‘Pinos’ (U.S. Plant Pat. No. 10,208), and a male parent, cultivar ‘Maribelle’ (unpatented in the United States). The new cultivar was discovered by the inventor in October 1996.

Asexual reproduction of the new cultivar by apical tip cutting and meristem tissue culture was performed in Oxnard, Calif. and has shown that the unique features of this new cultivar are stable and reproduced true to type on successive generations.

### SUMMARY OF THE INVENTION

The cultivar ‘GEDI ONE SAV’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘GEDI ONE SAV.’ These characteristics in combination distinguish ‘GEDI ONE SAV’ as a new and distinct Chrysanthemum:

1. Large, decorative type inflorescence;

2. Consistent flowering response to short days, blooming consistently after 49 days of short day length;

3. Free branching habit; and

4. Highly floriferous plants.

Plants of the new cultivar are similar to plants of the parent cultivar, ‘Pinos,’ in most horticultural characteristics, however plants of the new cultivar differed from plants of the cultivar ‘Pinos’ in the following characteristics:

1. The new cultivar is taller and wider;

2. The new cultivar has larger diameter blooms; and

3. The new cultivar has different color ray florets.

Plants of the new cultivar are also similar to plants of the parent cultivar, ‘Maribelle,’ in most horticultural characteristics, however plants of the new cultivar differed from plants of the cultivar ‘Maribelle’ in the following characteristics:

1. The new cultivar has more ray florets;

2. The new cultivar has a more round, uniform plant shape; and

3. The new cultivar naturally blooms 2 week earlier.

The new cultivar can be compared to the Chrysanthemum cultivar ‘Jenny Wren’ (disclosed in Plant Pat. No. 10,213). However plants of the new Chrysanthemum differed from the plants of the cultivar ‘Jenny Wren’ in the following characteristics:

1. The new cultivar has more ray florets;

2. The new cultivar has a more round plant shape and greater branching;

3. The new cultivar has a shorter internode length;

4. The new cultivar has a more flexible plant form; and

5. The new cultivar has more blooms per flowering branch.

### BRIEF DESCRIPTION OF THE PHOTOGRAPHS

The photograph shows a plant of ‘GEDI ONE SAV’ grown in a 1-gallon container. A single cutting was used in the pot.

### DETAILED BOTANICAL DESCRIPTION

In the following description, color references are made to The Royal Horticultural Society Colour Chart except where general terms of ordinary dictionary significance are used. The following observations and measurements describe plants grown in a commercial filed on Oxnard, Calif. in the month of September 2000. The growing conditions range from 60° to 65° F. at night to 70° to 85° during the day. Measurements and numerical values represent averages of typical flowering types.

Botanical classification: Dendranthema morifolium cultivar ‘GEDI ONE SAV’

Commercial classification: Garden-type Chrysanthemum

Parentage: Hybridization of female parent ‘Pinos’ (U.S. Plant Pat. No. 10,208) and male parent ‘Maribelle’ (unpatented in the United States)

### Propagation

Time to Rooting: 7 to 14 days at approximately 21° C.

Rooting habit: Fine, fibrous

### Plant

Growth Habit: Mounding herbaceous perennial

Height: Approximately 22 cm

Growth rate: Moderate

Branching Characteristics: Free Branching

Length of Lateral Branches: Approximately 18 cm

Number of leaves per lateral branch: Approximately 18

### Foliage

Leaf:

Arrangement.—Alternate single

Average length.—Approximately 5.4 cm

Average width.—l Approximately 3.4 cm

Shape of blade.—Ovate

Apex.—Acuminate

Base.—Attenuate

Attachment.—Stalked

Margin.—Serrate

Texture of top surface.—Lightly pubescent

Texture of bottom surface.—Lightly pubescent

Color.—young foliage upper side: Near 147A young foliage under side: Near 147A mature foliage upper side: Near 147A mature foliage under side: Near 147A venation upper side: Near 148B venation under side: Near 147B

Petiole:

Average length.—Approximately 2.0 cm

### Bloom

Inflorescence:

Flowering habit.—Induced by darkness period greater than 13.5 hours, approximately 49 days of appropriate day length required to induce and develop blooms

Inflorescenceform.—Decorative

Natural flowering season.—Approximately the first two weeks in September

Number of inflorescences per stem.—Approximately 18

Inflorescence diameter.—Approximately 4.7 cm

Infiorescence depth.—Approximately 2.0 cm

Inflorescence longevity on plant.—Approximately 24-27 days

Persistence.—Persistent

Ray Florets:

Appearance.—Matte

Texture.—Smooth

Average number per flower.—Approximately 190

Shape.—Cylindric

Margin.—Entire

Apex.—Obtuse

Length.—Approximately 0.2 cm

Width.—Approximately 0.5 cm

Color.—upper surface at first opening: Near 183A upper surface at maturity: Near 1 85A upper surface at fading: Near 1 82B under surface at first opening: Near 183C under surface at maturity: Near 184B under surface at fading: Near 1 82C

Disc florets:

Appearance.—Shiny

Texture.—Smooth

Average number per flower.—Approximately 1

Shape.—Cylindric

Apex.—Obtuse

Average length.—Approximately 0.2 cm

Average width.—Approximately 0.1 cm

Color.—at first opening: Near 154B at maturity: Near 2A at fading: Near 11A

Peduncle:

Length.—at terminal end (shortest): Approximately 2.5 cm at lateral end (longest): Approximately 2.8 cm

Strength.—Moderate

pi Color.—Near 146A

Habit.—Erect

Inflorescence bud:

Length.—Approximately 0.5 cm

Diameter.—Approximately 0.5 cm

Form.—Globular

Color.—Near 137A

Sepals:

Appearance.—Matte

Texture.—Lightly pubescent

Number.—Approximately 50 to 60

Shape.—Ovate

Margin.—Entire

Apex.—Acute

Color.—upper side: Near 137B under side: Near 137B

### Reproductive Organs

Ray Florets:

Pistillate.—Number of pistils per flower: 1 Stigma shape: 2 branched Stigma color: Near 1SA Style length: Approximately 0.1 cm

Disc Florets:

Pistillate.—Number of pistils per flower: 1 Stigma shape: Cylindric Style length: Approximately 0.2 cm

Staminate.—Number of stamens per flower: Approximately 5 Anther shape: Tubular Anther color: Near 15A Pollen color: Near 15A

### Other Characteristics

Disease Resistance: Neither resistance nor susceptibility observed

### ‘GEDI ONE SAV’ : Table of R.H.S. Color Codes

| | | | |
|---|---|---|---|
| FOLIAGE | Leaf | young foliage upper surface | Near 147A |
| | | young foliage under surface | Near 147A |
| | | mature foliage upper surface | Near 147A |
| | | mature foliage under surface | Near 147A |
| | | venation upper surface | Near 148B |
| | | venation under surface | Near 147B |
| BLOOM | Ray Florets | upper surface at first opening | Near 183A |
| | | upper surface at maturity | Near 185A |
| | | upper surface at fading | Near 182B |
| | | under surface at first opening | Near 183C |
| | | under surface at maturity | Near 184B |
| | | under surface at fading | Near 182C |
| | | Pistillate: Stigma | Near 15A |
| | Disc Florets | at first opening | Near 154B |
| | | at maturity | Near 2A |
| | | at fading | Near 11A |
| | | Staminate: Anther | Near 15A |
| | | Staminate: Pollen | Near 15A |
| | Peduncle | | Near 146A |
| | Inflorescence | | Near 137A |
| | Bud | | |
| | Sepals | upper surface | Near 137B |
| | | under surface | Near 137B |

What is claimed is:

1. A new and distinct variety of Chrysanthemum plant named ‘GEDI ONE SAV’ as herein illustrated and described.
